(19) United States
(12) Patent Application Publication
(10) Pub. No.: US 2018/0192933 A1
(43) Pub. Date: Jul. 12, 2018

(54) CONTAINER DEVICE FOR COLLECTING, STORING AND PROCESSING BLOOD OR A BLOOD COMPOUND

(71) Applicant: BIOTECHNOLOGY INSTITUTE, I MAS D, S.L., Vitoria (Alava) (ES)

(72) Inventor: Eduardo ANITUA ALDECOA, Vitoria (Alava) (ES)

(21) Appl. No.: 15/869,115

(22) Filed: Jan. 12, 2018

(30) Foreign Application Priority Data
Jan. 12, 2017 (ES) P 201730029

(51) Int. Cl. A61B 5/15

(52) U.S. Cl. CPC A61B 5/150755; A61M 1/3693; A61B 5/150351

(57) ABSTRACT

Container device (1) for collecting, storing and processing blood or a blood compound, which comprises a tube (10) with a connector (23) on its distal end (13), a piston set (30, 50) that can move through an interior space (14) of the tube (10), and a valve set (70, 90) preventing the flow of fluid through the distal opening (16) of the tube (10). The valve set (70, 90) is operable to allow the flow of the fluid through the distal opening (16). The device (1) enables vacuum conditions to be created in the tube (10) when it is going to be used, and enables the contents of the tube (10) to be extracted by operating the valve set (70, 90) without the need to use a needle.

### FIELD OF THE INVENTION

The invention relates to a container device for collecting, storing or processing blood or a blood compound. More particularly, the invention relates to a container device inside which a vacuum is created just before its use, and inside which blood or a blood compound is stored or processed, and from which it is possible to transfer the blood or compound to a second container, all of which can be carried out without the need to use a needle.

### PRIOR ART

The extraction of a small amount of blood from a human or animal patient is normally carried out using a small container and a butterfly needle. The container has an interior space in which there is a depression (commonly referred to as a “vacuum”), and a perforable end or cap. In turn, the butterfly needle consists essentially of a narrow tube ending in two needles, a first needle intended to be inserted into the patient's vein, and a second needle intended to pierce the cap of the container. When the end needles of the butterfly needle prick the vein and the container, communication is established between the vein and the interior of the container, and the blood is suctioned from the vein to the interior of the second container due to the pressure difference. The butterfly may also comprise a shutter for opening and closing the flow of fluid through the tube, enabling the practitioner to commence and finish the blood extraction by operating the shutter.

Once the blood extraction to the container has been completed, the blood is usually processed inside this container. For example, the container may be placed inside a centrifugal machine and centrifuged at a certain speed for a defined time in order to separate the blood into fractions (for example, a red cell fraction, a white cell fraction, and a fraction of platelet-rich plasma). During or after the blood processing, it is common to have to extract all or part of the contents of the container. To do so, a needle is usually inserted through the perforable cap, suctioning all or part of the contents of the container. Once the contents have been extracted, the needle is removed and the perforable cap once more seals the container. In other cases, the cap is removed and the needle or cannula is introduced into the container to suction the required fraction or fractions.

The use of needles involves certain risks. Obviously, there is the risk of the practitioner pricking him/herself with the needle. Furthermore, there is the risk of the needle being contaminated and this contamination being passed through the perforable cap and entering the container when the needle pierces the perforable cap.

On the other hand, containers used to extract blood are generally fitted with the aforementioned internal depression during production. In other words, the containers are marketed with internal “vacuum” conditions. However, over time, all plastic containers with an internal vacuum gradually lose the vacuum (i.e. there is a gradual increase in the internal pressure). Therefore, when using a plastic container with internal vacuum conditions that has been manufactured some time ago, there is a possibility that the container will not work properly, i.e., will not able to extract the necessary volume of blood.

The purpose of this invention is to solve at least one of the aforementioned problems, i.e., solve the problems derived from the use of needles while using a blood extraction container and/or solve the problem of the loss of internal vacuum conditions throughout the time during which the container is stored prior to its use. If possible, a container is also sought that avoids problems of contamination and manipulation of the blood and its derivatives (problems that are associated with the performance of processes in an open circuit in which the contents of the container are exposed to the atmosphere), avoiding the need to use laminar flow hoods.

### BRIEF DESCRIPTION OF THE INVENTION

The object of the invention is a container device for collecting, storing and processing blood or a blood compound. The device comprises a hollow tube, a piston set and a valve set. The tube comprises a tubular body and delimits an interior through space which ends in a proximal opening located at the proximal end of the tube and in a distal opening located at the distal end of the tube. At the distal end, the tube is fitted with a connector. The piston set, in turn, can move in the interior space of the tube and comprises a piston head and a handle, which can preferably be disconnected from the piston head and reconnected to the piston head. The piston head is positioned at a distal end of the piston set and comes into fluid-tight contact with the tubular body of the tube delimiting and isolating two regions in the interior space of the tube. The handle extends from the piston head and protrudes from the tube at the proximal end of the tube. The valve set, in turn, blocks the flow of fluid at a distal end of the interior space of the tube preventing the flow of fluid through the distal opening of the tube. The valve set is operable, such as by pressure or perforation from the exterior, to unblock the flow of fluid through the distal end of the interior space and allow the flow of fluid through said distal opening.

The device as per the invention allows extracting blood, storing blood, processing the extracted blood and delivering all or part of the processed blood into a container, without the need for needles. This increases the safety of the user, simplifies the execution of the process and reduces the risk of contamination of the biological substances involved. Furthermore, it allows for the extraction, storing, processing and introduction of the blood in a container to be carried out in a closed circuit, i.e. without the need to open the device or expose the blood or its derivatives to the atmosphere; this eliminates the need for a costly laminar flow installation.

### BRIEF DESCRIPTION OF THE FIGURES

The details of the invention can be seen in the accompanying figures, which do not intend to limit the scope of the invention:

FIG. 1 shows a top perspective view of a tube of a device as per an illustrative embodiment of the invention.

FIG. 2 shows a bottom perspective view of the tube of FIG. 1.

FIG. 3 shows a cross-sectional front elevation view of the tube of FIG. 1.

FIG. 4 shows a top perspective view of a handle of a device in accordance with an illustrative embodiment of the invention.

FIG. 5 shows a bottom perspective view of the handle of FIG. 4.

FIG. 6 shows a cross-sectional front elevation view of the handle of FIG. 4, wherein the section has been carried out in accordance with section plane 6-6 indicated in FIG. 4.

FIG. 7 shows a top perspective view of a piston head of a device in accordance with an illustrative embodiment of the invention.

FIG. 8 shows a bottom perspective view of the piston head of FIG. 7.

FIG. 9 shows a front elevation view of the piston head of FIG. 7.

FIG. 10 shows a cross-sectional side elevation view of the piston head of FIG. 7, wherein the section has been carried out in accordance with section plane 10-10 indicated in FIG. 9.

FIG. 11 shows a top perspective view of a septum of a valve set of a device in accordance with an illustrative embodiment of the invention.

FIG. 12 shows a bottom perspective view of the septum of FIG. 11.

FIG. 13 shows a front elevation view of the septum of FIG. 11.

FIG. 14 shows a cross-sectional side elevation view of the septum of FIG. 11, wherein the section has been carried out in accordance with section plane 14-14 indicated in FIG. 13.

FIG. 15 shows a top perspective view of a base of a valve set of a device in accordance with an illustrative embodiment of the invention.

FIG. 16 shows a bottom perspective view of the base of FIG. 15.

FIG. 17 shows a front elevation of the base of FIG. 15.

FIG. 18 shows a cross-sectional side elevation view of the base of FIG. 15, wherein the section has been carried out in accordance with section plane 18-18 indicated in FIG. 17.

FIG. 19 shows a top perspective view of a hood of a device in accordance with an illustrative embodiment of the invention.

FIG. 20 shows a bottom perspective view of the hood of FIG. 19.

FIG. 21 shows a front elevation of the hood of FIG. 19.

FIG. 22 shows a cross-sectional side elevation view of the hood of FIG. 19, wherein the section has been carried out in accordance with section plane 22-22 indicated in FIG. 21.

FIG. 23 shows a cross-sectional front elevation of the assembled device with the handle in an advanced position in accordance with an illustrative embodiment of the invention.

FIG. 24 shows an enlarged view of the distal end of the device of FIG. 23.

FIG. 25 shows six steps of an illustrative sequence of use of the device of FIG. 23.

FIG. 26 shows four remaining steps of said illustrative sequence of use of the device of FIG. 23.

FIG. 27 shows an enlarged view of the distal end of the device in the position of the fifth step of FIG. 25.

FIG. 28 shows an enlarged view of the proximal end of the tube, with the piston head clipped to this proximal end.

FIG. 29 shows an enlarged view of the distal end of the device in the position of the third step of FIG. 26.

FIG. 30 shows an enlarged view of the upper part of a device in accordance with a second illustrative embodiment of the invention.

FIG. 31 shows seven steps of another illustrative sequence of use of the device of FIG. 23 without the hood.

FIG. 32 shows six steps of yet another illustrative sequence of use of the device of FIG. 23 without the hood.

FIG. 33 shows five subsequent steps following those of FIG. 32.

FIG. 34 shows seven steps of another illustrative sequence of use of the device of FIG. 23 without the hood.

FIG. 35 shows seven subsequent steps following those of FIG. 34.

FIG. 36 shows seven steps of yet another illustrative sequence of use of the device of FIG. 23 without the hood.

FIG. 37 shows seven subsequent steps following those of FIG. 36.

FIG. 38 shows eight steps of yet another illustrative sequence of use of the device of FIG. 23 without the hood.

### BRIEF DESCRIPTION OF THE FIGURES

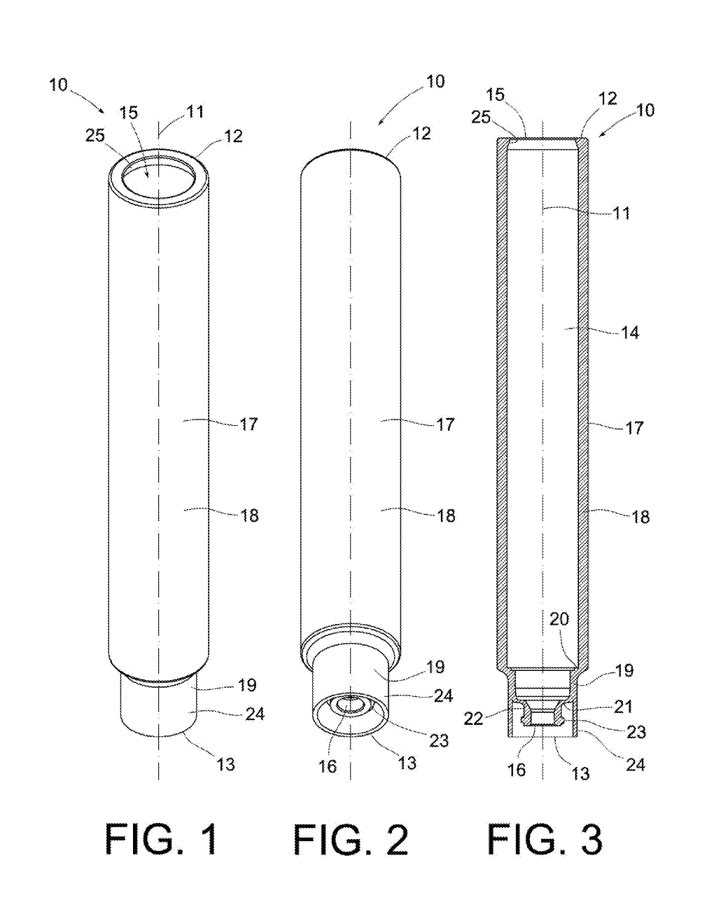

FIGS. 1 to 22 show the different parts or components of a container device (1) in accordance with an illustrative embodiment of the invention. This device can be used to collect, store or process blood or a blood compound, as will be explained in greater detail herein.

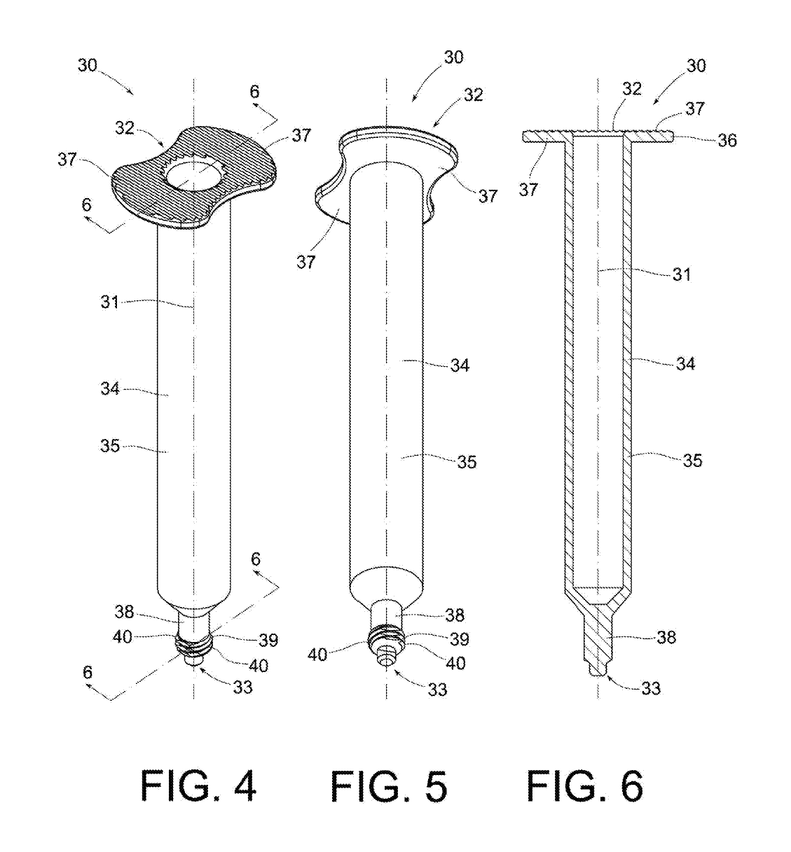

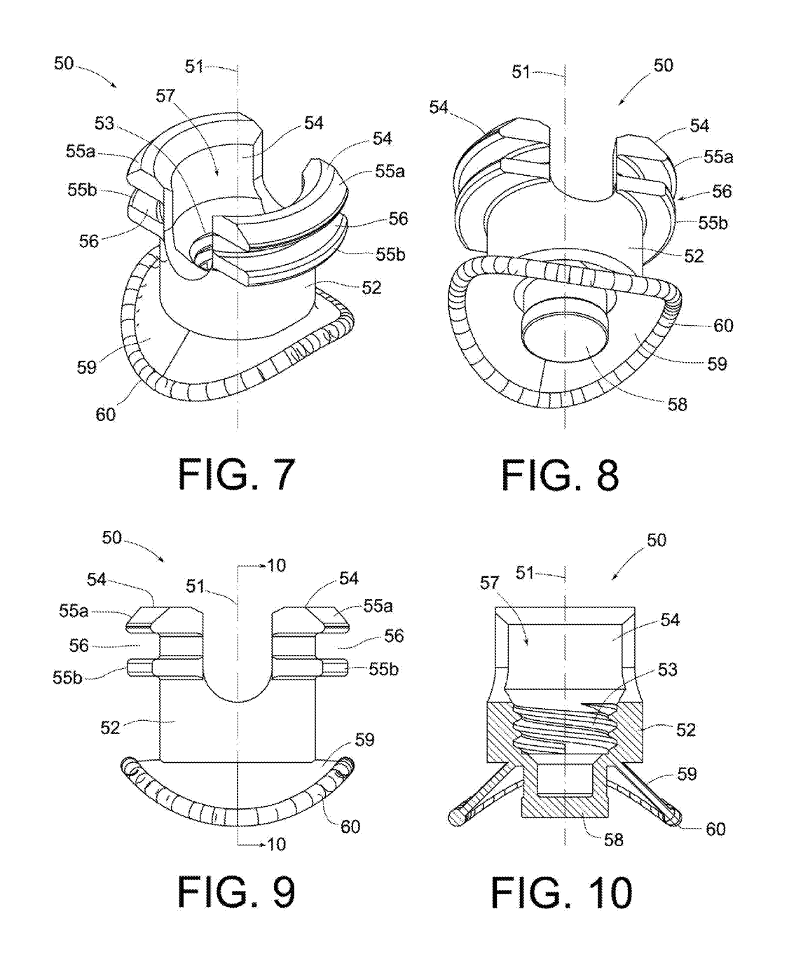

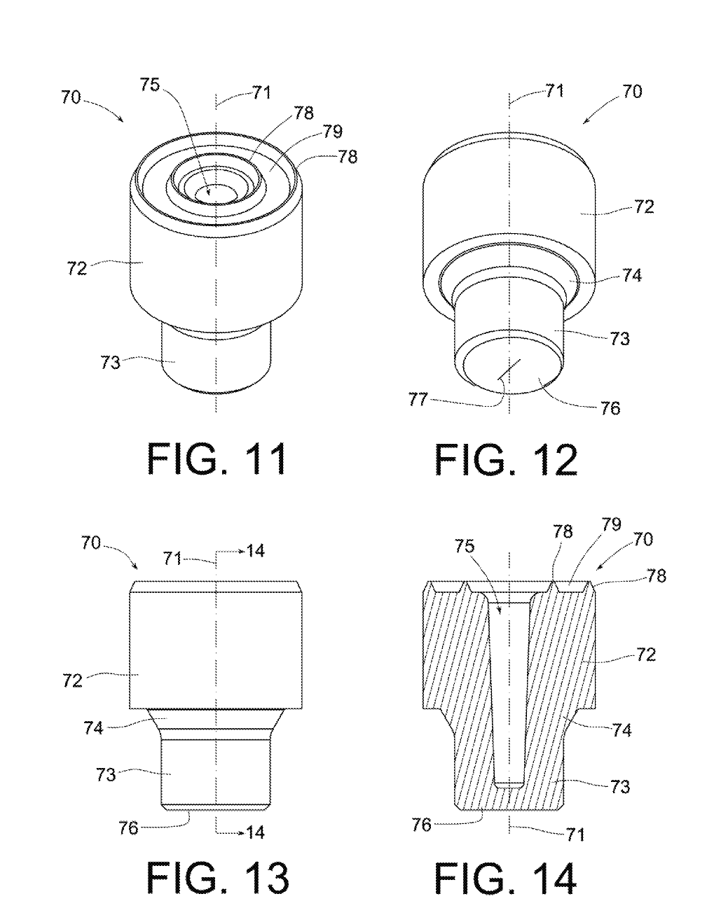

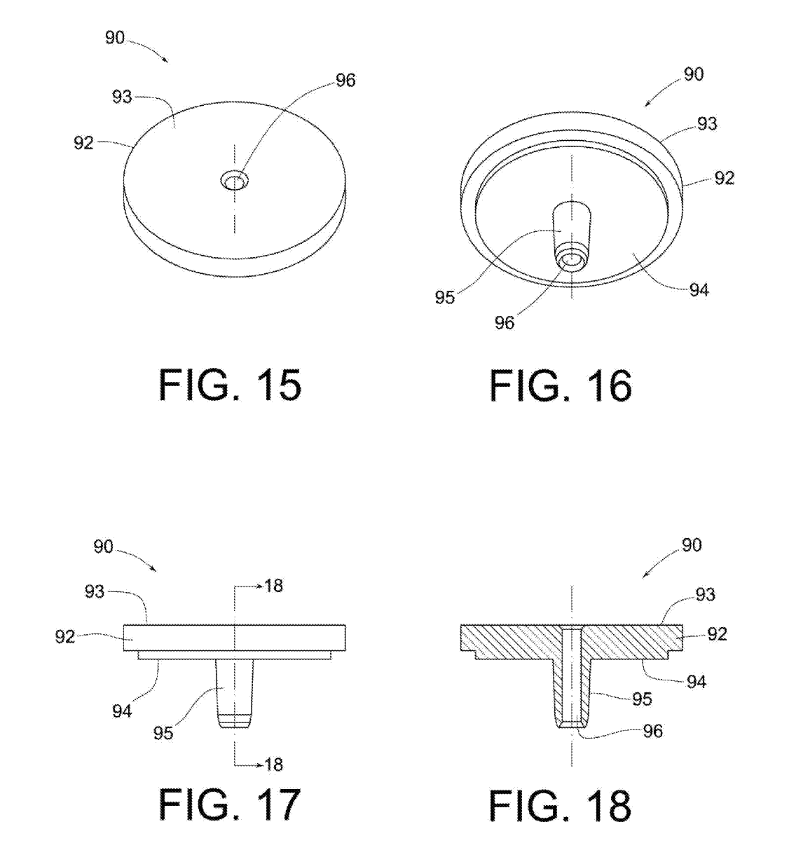

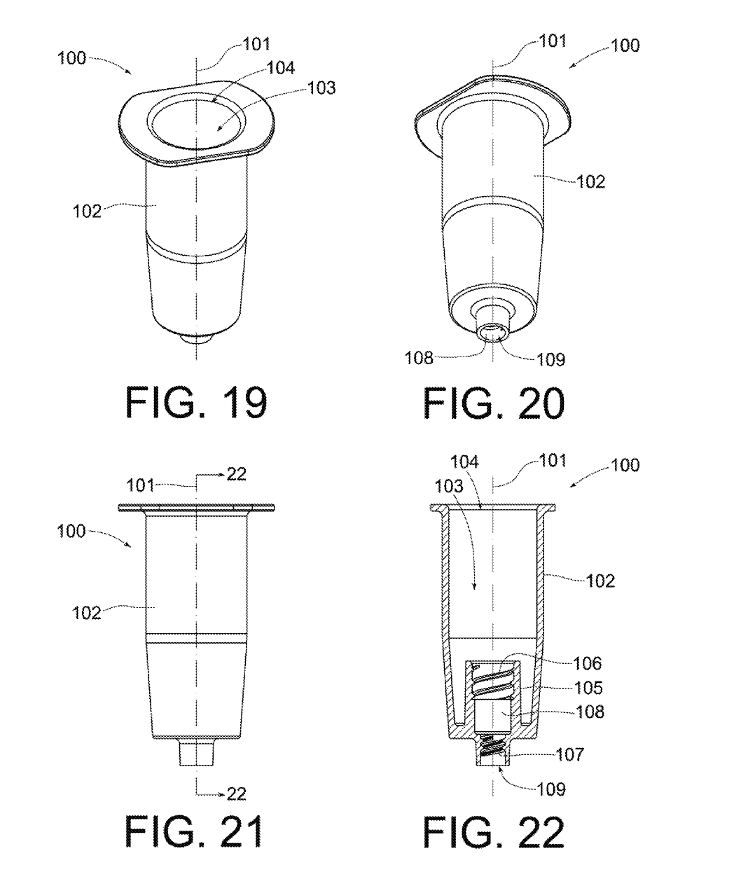

The container device (1) of the present example comprises a tube (10), shown in FIGS. 1 to 3, inside which a piston set (30, 50) moves. The piston set (30, 50) is made up of a piston shaft or handle (30), shown in FIGS. 4-6, and a piston head (50), shown in FIGS. 7-10. As will be seen in greater detail herein, a distal end of the tube (10) is closed by a deformable and perforable valve set (70, 90), which is made up of a septum (70) and a base (90), shown respectively in FIGS. 11-14 and in FIGS. 15-18. The device (1) may also comprise a hood (100), shown in FIGS. 19-22.

FIGS. 1-3 show the tube (10) of the device (1) in detail. The tube (10) is formed along a longitudinal axis (11) and has a proximal end (12) and a distal end (13). The tube (10) is hollow, presenting an interior space (14) that extends along the whole length of the tube (10) from the proximal end (12) to the distal end (13) and ends in a proximal opening (15) and in a distal opening (16). The tube (10) is essentially comprised of a tubular body (17) formed by a cylindrical wall (18) which surrounds the interior space (14). Following the wall (18), there is a second cylindrical wall (19) which has a smaller diameter than the wall (18) and also surrounds the interior space (14). Between this wall (18) and the second wall (19) there is a transition shoulder (20). After the second wall (19) and behind a second transition shoulder (21) there is a third essentially cylindrical wall (22) that is fitted with a connector (23). In some embodiments, such as the one shown in the figures, the connector (23) is a threaded connector. This connector (23) is preferably able to connect to a syringe; for example, the connector (23) can be a threaded male “luer-lock” termination, of the type defined in the ISO 594, DIN/EN 1707:1996 and 20594-1:1993 standards. A generally cylindrical, encircling wall or skirt (24) extends distally from the second wall (19), surrounding the third wall (22) and the connector (23). The skirt (24) may be built as a continuation of the second wall (19), i.e. having the same shape as the second wall (19). The function of the skirt (24) is to protect the connector (23) from any contact with other bodies during the handling of the device (1), for which it is preferable for the skirt (24) to be longer than the connector (23), i.e. it preferably protrudes distally with respect to the connector (23), such as in the illustrated embodiment. Finally, at the proximal end (12) of the tube (10) there is a lip (25) which fully surrounds the proximal opening (15) and causes the proximal opening (15) to have a smaller diameter than the inner diameter of the tubular body (17).

FIGS. 4-6, in turn, show the piston axis or handle (30). This handle (30) is formed around a longitudinal axis (31) and has a proximal end (32) and a distal end (33). The handle (30) is essentially comprised of a tubular body (34) formed by a substantially cylindrical wall (35). At the proximal end (32) of the handle (30), the handle (30) is fitted with a grip or gripping area (36), in this case in the form of two flat parts (37) that extend transversally with respect to the longitudinal axis (31) and protrude towards the sides of the tubular body (34) in order to help the user pull or push the handle (30) in the direction of the longitudinal axis (31), as will be seen herein. At the distal end (33) of the handle (30) there is a neck (38) that is narrower (i.e. provided with a smaller width or diameter) than the tubular body (34). A connector (39), such as a male threaded termination, is provided on the neck (38) for disconnectably connecting the piston head (50), as will be seen in greater detail herein. The connector (39) of the present embodiment is a male threaded termination with threads that do not extend along a full 360-degrees turn about the longitudinal axis (31), but rather extend along two opposed, threaded lugs (40), which is why this threaded termination cannot be seen in the transversal section in FIG. 6.

FIGS. 7-10, in turn, illustrate the piston head (50). This piston head (50) is formed along a longitudinal axis (51) and comprises a body (52) having an internal connector (53). In the present case, the connector (53) is a female, threaded connector. Both the body (52) and the connector (53) are formed along the longitudinal axis (51). Two protrusions (54), which are slightly elastic (i.e. have a tendency to recover their rest position, shown in the figure), extend proximally from a proximal end of the body (52) and at opposite sides of the body (52). Two transversal protrusions (55) extend radially outwards from each protrusion (54). The two transversal protrusions (55) are slightly separated from each other so that a space (56) is provided therebetween, wherein the space (56) is oriented radially or transversally outwards. The piston head (50) comprises an interior cavity (57) which extends between the two protrusions (54) and through the connector (53). This interior cavity (57) is blind. i.e. closed at the distal end by a wall (58). The piston head (50) further comprises a skirt (59) which extends distally and flares outward from the body (52) and, in the present embodiment, has an undulated edge. This edge (60), as will be seen in detail hereinafter, is configured to come into contact with the wall (18) of the tubular body (17) of the tube (10) along the whole perimeter of the edge (60) in order to fully seal the piston head (50) against said wall (18).

FIGS. 11-14 show several views of the septum (70) of the valve set (70, 90). The septum (70) is formed along a longitudinal axis (71) and comprises a first portion (72) and a second portion (73) that is narrower than and extends distally from the first portion (72). In the present embodiment, the first portion (72) and the second portion (73) are cylindrical and are connected by a conical part (74) or portion having a decreasing width. The septum (70) has an interior cavity (75) which extends from the proximal end of the first portion (72), through the first portion (72) and the second portion (73), and is closed at its distal end by a wall (76). As can be seen, the interior cavity (75) may be slightly conical. As can be observed in FIG. 12, a slit or cut (77) may extend through the wall (76). The cut (77) is normally closed, preventing the flow of fluid through the wall (76). The wall (76) may be deformed in such a way that, when the wall (76) is deformed, the cut (77) opens, allowing the flow of fluid to and from the interior cavity (75). Alternatively or additionally to the cut (77), the wall (76) may be perforable to allow the insertion of a needle through the wall (76) and into the interior cavity (75). At the proximal end, the septum (70) is provided with circular ribs or protrusions (78) that are slightly flexible and positioned in the direction of the longitudinal axis (71). Spaces (79) are defined between the ribs or protrusions (78).

FIGS. 15 to 18 show the base (90) of the valve set (70, 90). The base (90) is formed along a central longitudinal axis (91) and comprises a disc (92) arranged concentrically to the central longitudinal axis (91). The disc (92) has a proximal side (93) and a distal side (94). A neck (95) extends distally from the distal side (94) of the disc (92). This neck (95) is slightly conical in order to be press fitted inside the interior cavity (75) of the septum (70). The base (90) further comprises a longitudinal, through interior cavity (96), which extends completely through the base (90) in the direction of the longitudinal axis (91). This interior cavity (96) is arranged around the longitudinal axis (91).

Finally, FIGS. 19 to 22 show the hood (100), which is arranged along a longitudinal axis (101) and comprises a hollow main body (102) having an interior space (103) sized to receive the tubular body (17) of the tube (10) of FIGS. 1 to 3. At a proximal end of the hood (100), there is an opening (104) to allow for the insertion of the tube (10) in the interior space (103). At a distal end of the hood (100), a neck (105) extends towards the interior space (103). This neck (105) has a first connector, which in this case is a first threaded connection (106), for the connection of the connector (23) of the tube (10). The first threaded connection (106) of this embodiment is female, and preferably a threaded female “luer-lock” termination, similar to that defined in the ISO 594, DIN/EN 1707:1996 and 20594-1:1993 standards. Furthermore, a second threaded connection (107) is positioned distally with respect to the first threaded connection (106). This second threaded connection (107) is preferably a female threaded connection configured to be connected to a male threaded connection (126) of a butterfly needle (120), as shown in FIG. 24. An interior duct (108) extends through the neck (105), the first threaded connection (106) and the second threaded connection (107) and up to a distal opening (109).

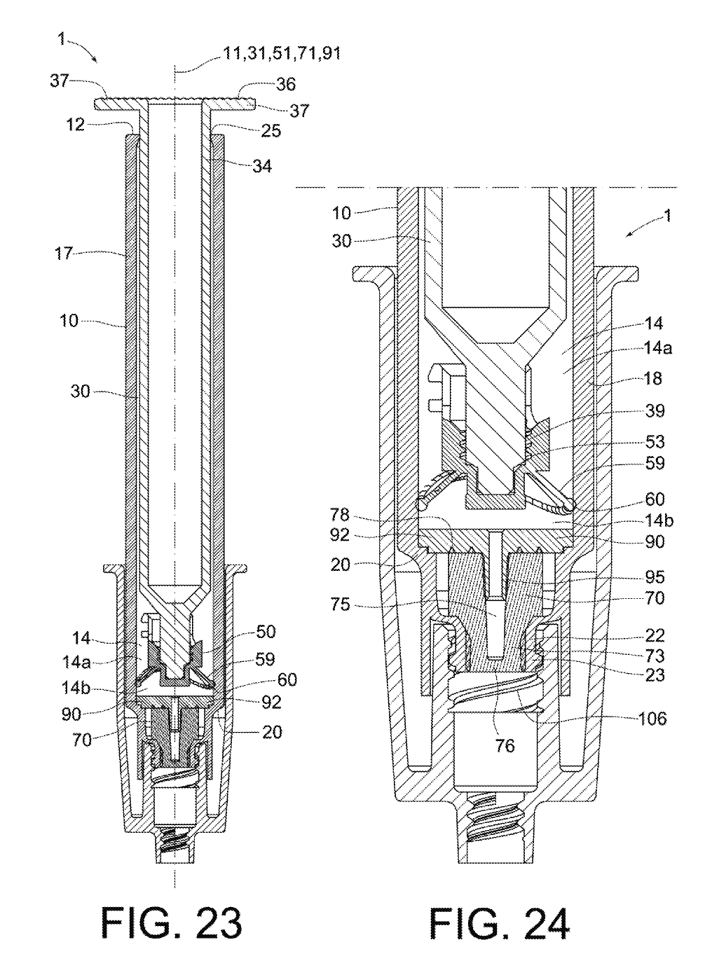

FIGS. 23 and 24 show the device (1) in an assembled state. As can be seen, the valve set (70, 90) is placed inside the tube (10), in the interior space (14). The second portion (73) of the septum (70) of the valve set (70, 90) is in the interior space (14), surrounded by—and preferably adjusted against—the third wall (22) of the tube (10), so that the septum (70) remains affixed to this third wall (22) by friction. In turn, the base (90) is coupled to the septum (70) by having the neck (95) of the base (90) introduced and retained by friction within the interior cavity (75) of the septum (70). The septum (70) is coupled to the base (90) with a slight pressure towards the base (90) such that the circular protrusions (78) of the septum (70) are in fluid-tight contact with the distal side (94) of the base (90), producing a seal that guarantees fluid-tightness with the base (90). The disc (92) of the base (90) is supported on the shoulder (20) of the tube (10).

The handle (30) is inserted inside the tube (10), in the interior space (14) of the tube (10). The connector (39) (in this case, a threaded termination) of the handle (30) is connected to the connector (53) (in this case, a threaded connector) of the piston head (50) in such a way that it can be disconnected (in this case by unthreading). As the handle (30) and the piston head (50) are connected, they can move together or in unison along the interior space (14) of the tube (10). The proximal end of the handle (30) protrudes from the proximal end (12) of the tube (10), such that the gripping area (36) remains outside the tube (10) and is accessible for the user to push or pull the handle (30). The lip (25) of the proximal end (12) of the tube (10) adjusts against the tubular body (34) of the handle (30).

In the situation in FIGS. 23 and 24, the edge (60) of the skirt (59) of the piston head (50) is in fluid-tight contact with the wall (18) of the tubular body (17) of the tube (10) along the whole perimeter of the edge (60), providing an absolute sealing of the piston head (50) against this wall (18) and separating the interior space (14) of the tube (10) into two chambers or regions (14a, 14b). The region (14b) located distally (in front) of the piston head (50) is sealed or fluid-tightly closed by the piston head (50), by the wall (18) of the tube (10) and by the valve set (70, 90), and more particularly by the wall (76) of the septum (70) of the valve set (70, 90) which closes the interior cavity (75) of the septum (70).

In the situation in FIGS. 23 and 24, the handle (30) is in an advanced position with respect to the tube (10), i.e. inserted as far as possible or almost as far as possible inside the tube (10). If the user proceeds to pull the handle (30) rearwards, exerting an appropriate force against the flat parts (37) of the handle (30) to attempt extract it from the tube (10), the handle (30) begins to move proximally, increasing the volume of the region (14b) located in front of the piston head (50) and reducing the volume of the region (14a) located behind the piston head (50). The air from the region (14a) can pass between the lip (25) and the tubular body (34) and exit towards the exterior of the tube (10), allowing the handle (30) to move rearward; this air that exits the interior space (14) of the tube (10) between the lip (25) and the tubular body (34) of the handle (30) prevents the entry of bacteria and contaminating agents towards the interior space (14) of the tube (10). In turn, the increase in the volume of the fluid-tight region (14b) causes a depression in this region (14b). By making the handle (30) move sufficiently rearward, there comes a moment, illustrated in FIG. 28, in which the rear protrusion (55a) of the piston head (50) moves beyond the lip (25) of the tube (10) and the lip (25) is received in the space (56) between the protrusions (55a, 55b) causing the piston head (50) to become dipped to the proximal end (12) of the tube (10).

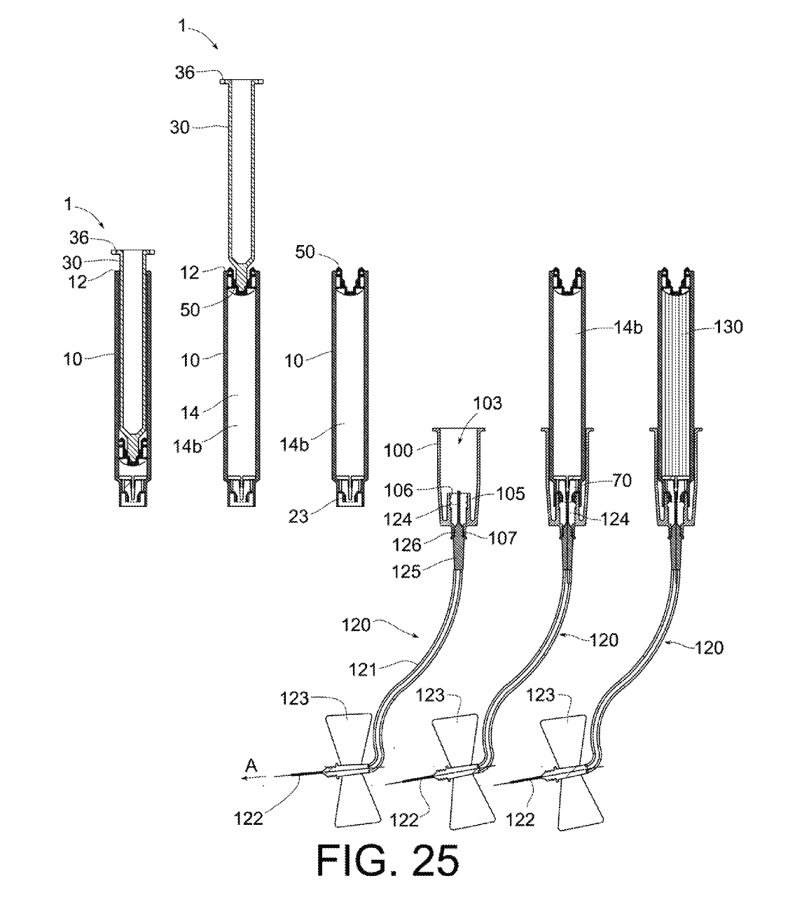

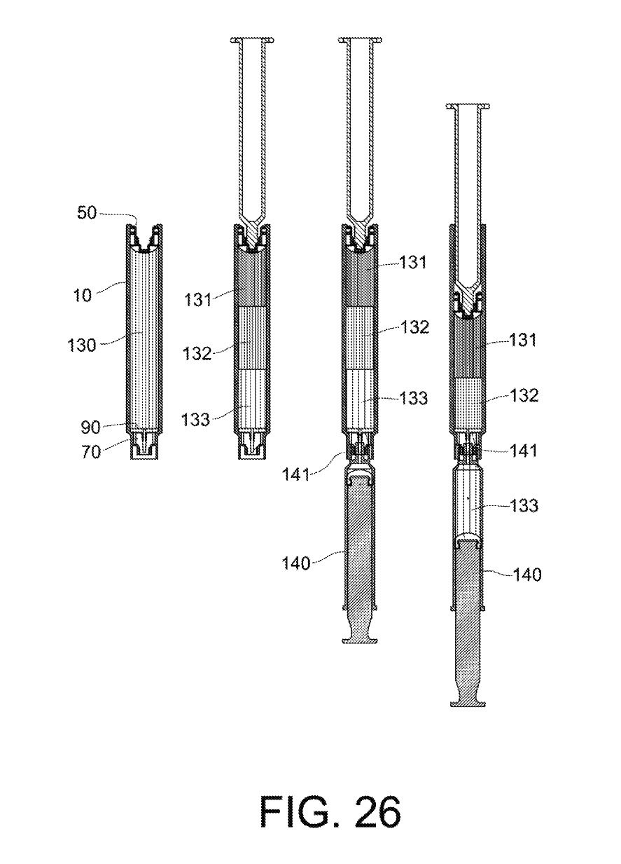

FIGS. 25 and 26 show a sequence of steps as per an example of use of the aforementioned device (1), where FIG. 25 shows six steps and FIG. 26 illustrates four steps of the sequence.

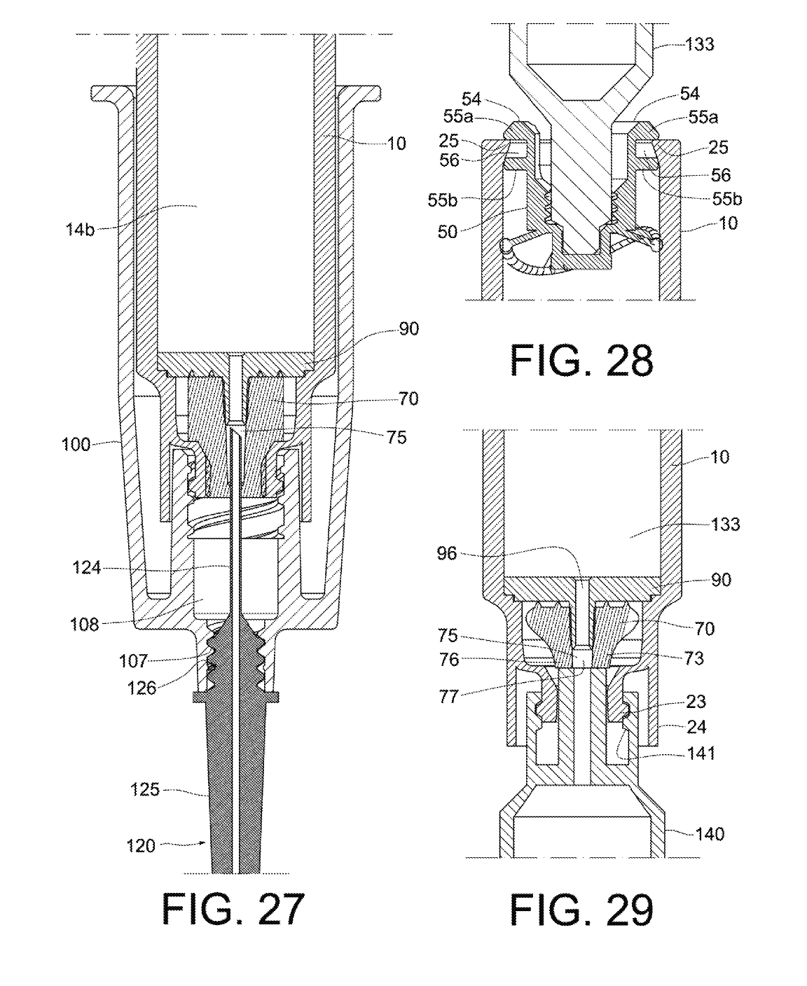

As can be seen in FIG. 25, the sequence of use begins with the device (1) in the situation of FIG. 23, i.e. with the handle (30) in an advanced position inside the tube (10). Then, in a second step, the user pulls the gripping area (36) of the handle (30) and causes the handle (30) to displace rearward inside the tube (10) until the piston head (50) is clipped to the proximal end (12) of the tube (10) (as previously described with reference to FIG. 28) and a depression has been formed in the region (14b). In a third step, the user turns the handle (30) with respect to the longitudinal axis (31), unthreading the connector (39) of the handle (30) from the connector (53) of the piston head (50), and proceeds to remove the handle (30), leaving the device (1) in a configuration in which it resembles a conventional vacuum tube, of the type that is used to remove relatively small amounts of blood from a human or animal body. Then, in a fourth step of the sequence, a butterfly needle (120), of the type known in the art, is provided. The butterfly needle (120) comprises a flexible tube (121), at one end of which there is a first needle (122) accompanied by a flat part or a butterfly (123), and at the opposite end of which there is a second needle (124) which extends from a connector (125) that has a male threaded connection (126). In this fourth step of the sequence, the first needle (122) is inserted into a patient's vein, pressing the butterfly (123) against the patient's skin to stabilize the first needle (122), and the second needle (124) is inserted in the interior duct (108) of the hood (100) whilst the threaded connection (126) of the connector (125) is threaded into the second threaded connection (107) of the hood (100). Then, in the fifth step of FIG. 25 (illustrated in greater detail in the enlarged view in FIG. 27) the device (1), which has interior vacuum conditions (i.e. which has a depression in the region (14b)) is inserted into the interior space (103) of the hood (100) and the connector (23) of the tube (10) is threaded to the first threaded connection (106) of the hood (100), causing the second needle (124) of the butterfly needle (120) to either perforate the wall (76) of the septum (70) of the valve set (70, 90) or to pass through the cut (77). In consequence, the second needle (124) becomes communicated with the interior cavity (75) of the septum (70) and with the interior cavity (96) of the base (90). In this way, a fluid passageway is established between the patient's vein and the region (14b) and, due to the pressure difference, blood starts flowing from the vein towards the region (14b). Eventually, the situation in the final step of FIG. 25, in which the region (14b) has been filled with blood (130), is reached.

In a further step (first step of FIG. 26), the user unthreads and disconnects the butterfly needle (120) from the tube (10), thereby obtaining a closed tube (10) that is filled with blood (130) and is ready to be processed. For example, processing may consist in centrifuging the tube in a centrifugal machine to separate the blood (130) into two or more fractions (131, 132, 133) as is well known in the prior art of medicine and odontology. Once the blood is separated into fractions (131, 132, 133), the user proceeds with the second step of FIG. 26, which consists in reconnecting the handle (30) to the piston head (50) by threading the connector (39) of the handle (30) to the connector (53) of the piston head (50). Then, as shown in the third step of FIG. 26, the user proceeds to connect a conventional syringe (140), of the type which comprises a female “luer-lock” threaded connection (141) at its distal end, by threading this female “luer-lock” threaded connection (141) to the male threaded “luer-lock” connector (23) of the tube (10). As can be seen in the enlarged view of FIG. 29, in connecting the threaded connection (141) to the tube (10), the threaded connection (141) pushes the septum (70) of the valve set (70, 90) inwards, causing the compression of the second portion (73) and causing the cut (77) to open forming fluid passageway for the fluid to pass through the wall (76) of the septum (70), thus leaving the interior of the syringe (140) in communication with the region (14b) of the device (1) through the open cut (77), the interior cavity (75) and the interior cavity (96). Then, as shown in the final step of FIG. 26, the user pushes the handle (30) with a certain force, releasing the clipping between the protrusions (55a, 55b) of the piston head (50) and the lip (25) of the tube (10). It could also happen that the clipping between the protrusions (55a, 55b) of the piston head (50) and the lip (25) of the tube (10) has already been released due to the centrifugal force during the centrifugation process. By continuing to push the handle (30), the handle (30) and the piston head (50) move forward along the interior space (14) of the tube (10), pushing the desired fraction or fractions (133, 132, 131) so that they transfer partially or entirely to the syringe (140) (for example, in the illustrated method, only the full front fraction (133) is transferred). Once the desired fraction or fractions (133, 132, 131) have been transferred, the user can unthread the syringe (140) from the device (1) and use the fraction or fractions (133, 132, 131) (in this case, the fraction (133)) contained in the syringe (140) for diverse medical applications. Note that in other examples of use of the device (1), a same syringe (140) can receive the contents of more than one device (1).

In summary, the device (1) as per the invention allows blood to be extracted, the extracted blood to be processed and all or part of the processed blood to be introduced into a conventional syringe, without requiring the use of needles or exposing the contents of the device to the exterior. This increases the safety of the user, simplifies the performance of the process and reduces the risk of contamination of the biological substances involved.

As an alternative to what is illustrated in the sequence of FIGS. 25 to 26, a conventional hood of the type that does not have a first threaded connection (105) can be used instead of the hood (100). In this case, the hood would be fixed by pressure or friction to the skirt (24) of the tube (10) and/or to the surface of the tubular body (17) of the tube (10). I.e., the hood (100) described in this document is optional and the device (1) can be used with conventional blood extraction hoods. However, the hood (100) described in this document is advantageous because it allows the exact depth of insertion of the needle to be selected. Furthermore, by turning the first threaded connection (106) of the hood (100) with respect to the tube (10), the valve set (70, 90) can be gradually opened, allowing to regulate the flow of the fluid passing through the valve set (70, 90).

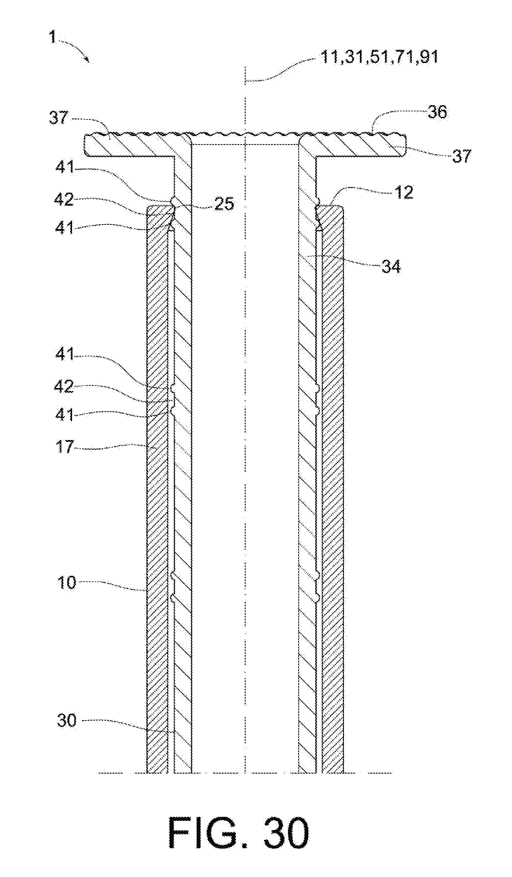

FIG. 30 shows an alternative embodiment of the invention, in which the tubular body (34) of the handle (30) comprises some protuberances (41) protruding radially from the tubular body (34). As can be seen, there is a retention valley or space (42) between two consecutive protuberances (41), wherein the space (42) is preferably sized such that the lip (25) is retained and relatively snugly fit between these two consecutive protuberances (41) and within the space (42), retaining the handle (30) so that it does not move longitudinally unless the user pulls or pushes the handle (30) with a force greater than a predetermined threshold. As can be seen in the figure, the protuberances (41) are preferably organised in pairs, the handle (30) having at least two pairs of protuberances (41). Between each pair of protuberances (41) there is a separation that is larger than the length of the space (42). In this way, the handle (30) provides at least two retention spaces (42) for the lip (25). The user can pull the handle (30) and, as the handle (30) is removed from the tube (10), the lip (25) moves along the tubular body (34) and is retained in each space (42), whilst the device makes a slight clicking sound whenever the lip (25) exceeds a protuberance (41) and enters inside the space (42). In short, this embodiment allows the handle (30) to be adjusted in different discrete positions with respect to the tube (10), whereby each position corresponds to a specific volume of the vacuum-provided chamber (14b). The adjustment is highly intuitive thanks to the successive audible clicks being emitted whenever a discreet position is reached.

Finally, in alternative embodiments of the invention, the piston head (50) and the handle (30) may be formed into a single piece, which can be manufactured for instance by injection moulding.

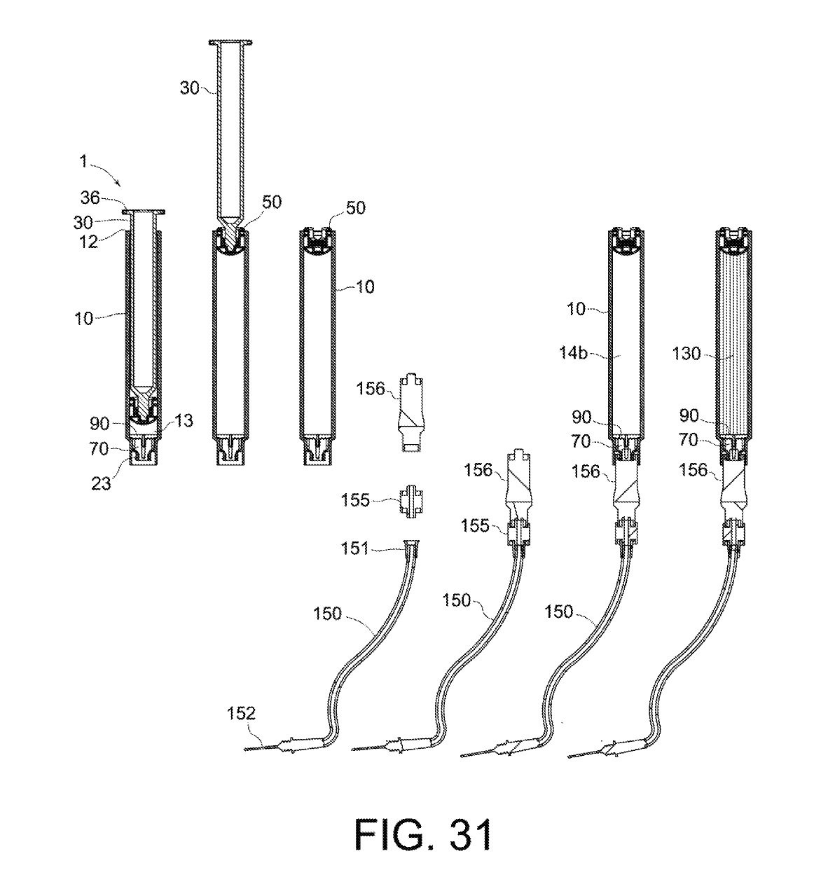

FIG. 31 shows a sequence of steps in accordance with a further illustrative use of the device (1). The first three steps of the sequence are the same as the first three steps of FIG. 25 and allow to configure the device (1) to have an internal depression or vacuum, a fluid-tight closure on the proximal end (12) and a valve set (70, 90) arranged blocking the flow of fluid at a distal end (13). Next, in a fourth step of the sequence, a butterfly needle (150) is obtained, having a female termination (151), a male-male connector (155) and a male-female valve (156), all of which are commercially available products. In a fifth step of the sequence, one end of the male-male connector (155) is connected to the male-female valve (156) and the other end is connected to the butterfly needle (150). Then, in a sixth step of the sequence, the first needle (152) of the butterfly needle (150) is inserted in a patient's vein and the connector (23) of the tube (10) is connected to the male-female valve (156) which is connected to the butterfly needle (150). The latter causes the valve set (70, 90) and the valve (156) to open and, in consequence, a fluid passageway to form between the patient's vein and the region (14b) of the tube (10), flow starting to flow towards the region (14b) due to pressure difference. Eventually, the situation of the seventh step of FIG. 31, in which the region (14b) has filled with blood (13), is reached. The procedure may then continue with the steps of FIG. 26, which have been described heretofore.

Thus, in the sequence of FIG. 31, a valve (156) having a female-male luer-lock connection (which is commercial) and a male-male connector (which is also commercial) are used instead of the hood (100) of FIGS. 19-22 and 25. Replacing the hood (100) with the valve (156) leads to an increase in fluid-tightness within the system. The blood remains within the butterfly needle (150) isolated from the outside. When the valve (156) of the butterfly needle (150) is connected to the valve set (70, 90) of the tube (10), the blood enters the tube (10) without having had a direct contact with the outside.

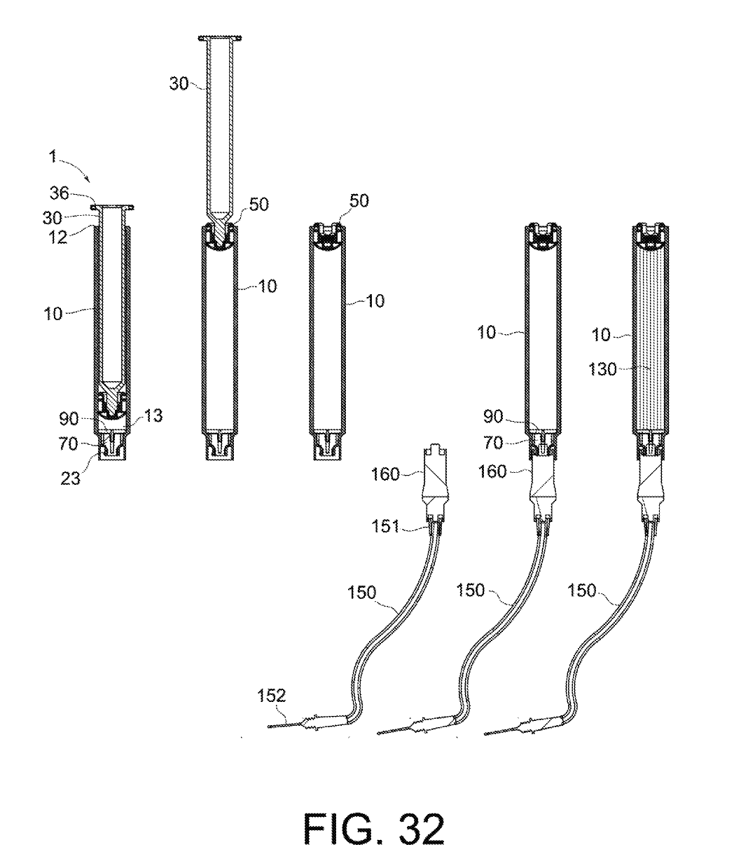

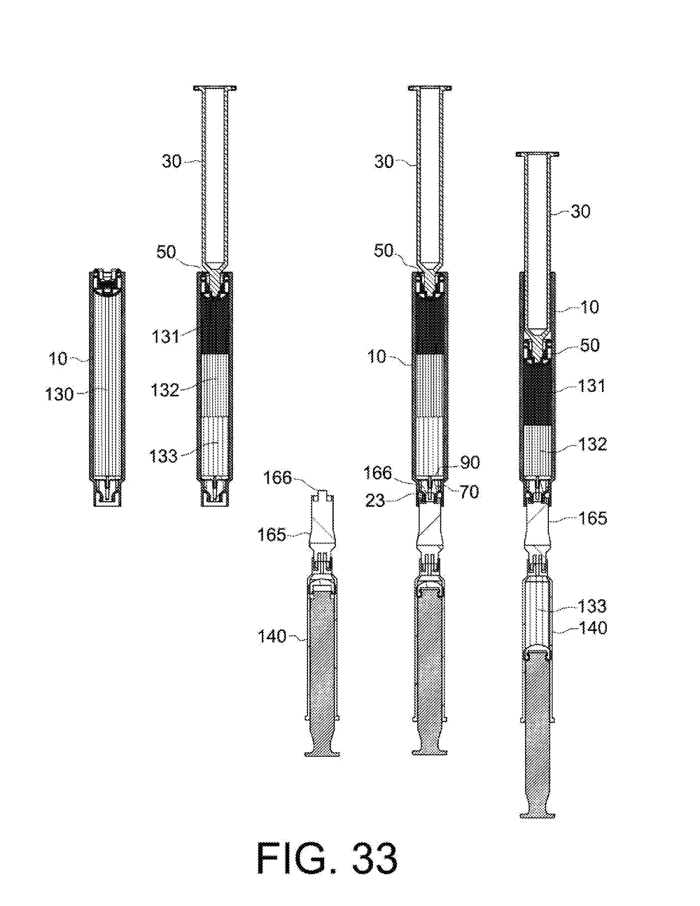

FIG. 32 shows a sequence of steps in accordance with a further use of the device (1). This sequence is almost identical to the sequence of FIG. 31, except for the fact that the butterfly needle (150) is provided with a male-male valve (160), i.e. a male connection capable of directly connecting to the butterfly needle (150) without the need for an adapter. Having the valve (160) connect directly to the butterfly needle (150) is advantageous in that, in this particular location, the system is subjected to no contamination as the inside of the butterfly needle (150) is isolated and the fluid passageway towards the tube (10) is opened only when the two valves (160; 70, 90) are connected to one another (fifth step of FIG. 32), thereby allowing the blood to remain isolated from the outside at all times. Similarly to the method of FIG. 31, the method of FIG. 32 can be followed by the steps of FIG. 26, which have been described heretofore. Alternatively, in another example of use of the device in accordance with the invention, the process of FIG. 32 may be followed by the steps of FIG. 33. In other words, after extracting blood as shown in FIG. 32, once the tube (10) filled with blood is obtained (first step of FIG. 33), the tube (10) is centrifuged to separate the blood in two or more fractions (131, 132, 133). Once the blood has been separated into fractions (131, 132, 133), the user proceeds with the second step of FIG. 33, which consists in re-connecting the handle (30) to the piston head (50). Next, as shown in the third step of FIG. 33, the user takes a commercial syringe (140) provided with a valve (165) having a female-male luer-lock connection (i.e. providing a male luer-lock connection (166) it its distal end) to increase fluid-tightness. Next, as shown in the fourth step, the male luer-lock connection (166) of the valve (165) of the syringe (140) is inserted in the threaded male “luer-lock” connector (23) of the tube (10), and the male luer-lock connection (166) pushes the septum (70) of the valve set (70, 90) inwards, opening the valve set (70, 90) and causing the formation of a fluid passageway through the valve set (70, 90). Similarly, connecting the valve (165) to the tube (10) causes the valve (165) to open and allow fluid to pass therethrough. Next, as shown in the last step of FIG. 33, the user pushes the handle (30) with a certain force causing the clipping between the piston head (50) and the tube (10) to end, if not previously unclipped, and the handle (30) to move forward pushing the desired fraction or fractions (133, 132, 132), partially or in their entirety, to cause a transfer thereof into the syringe (140) for further medical use.

In other words, the steps of FIG. 33 are similar to those of FIG. 26, with the exception that a female-male valve (165) is added to the fractioning syringe (140). This valve (165) can come incorporated to the syringe (140) and packaged with the syringe (140), in which case the interior of the syringe (140) remains sterilized even after opening the packaging. Furthermore, the transfer is carried out with no communication with the outside and thus preventing any kind of dripping when the syringe (140) is disconnected from the tube (10). Thus, by combining the valve (160) of the extraction butterfly needle (150) (FIG. 32) with the valve (165) of the fractioning needle (140) (FIG. 33), the blood and plasma are never exposed to the outside, thus allowing the system to be not only closed but also hermetic.

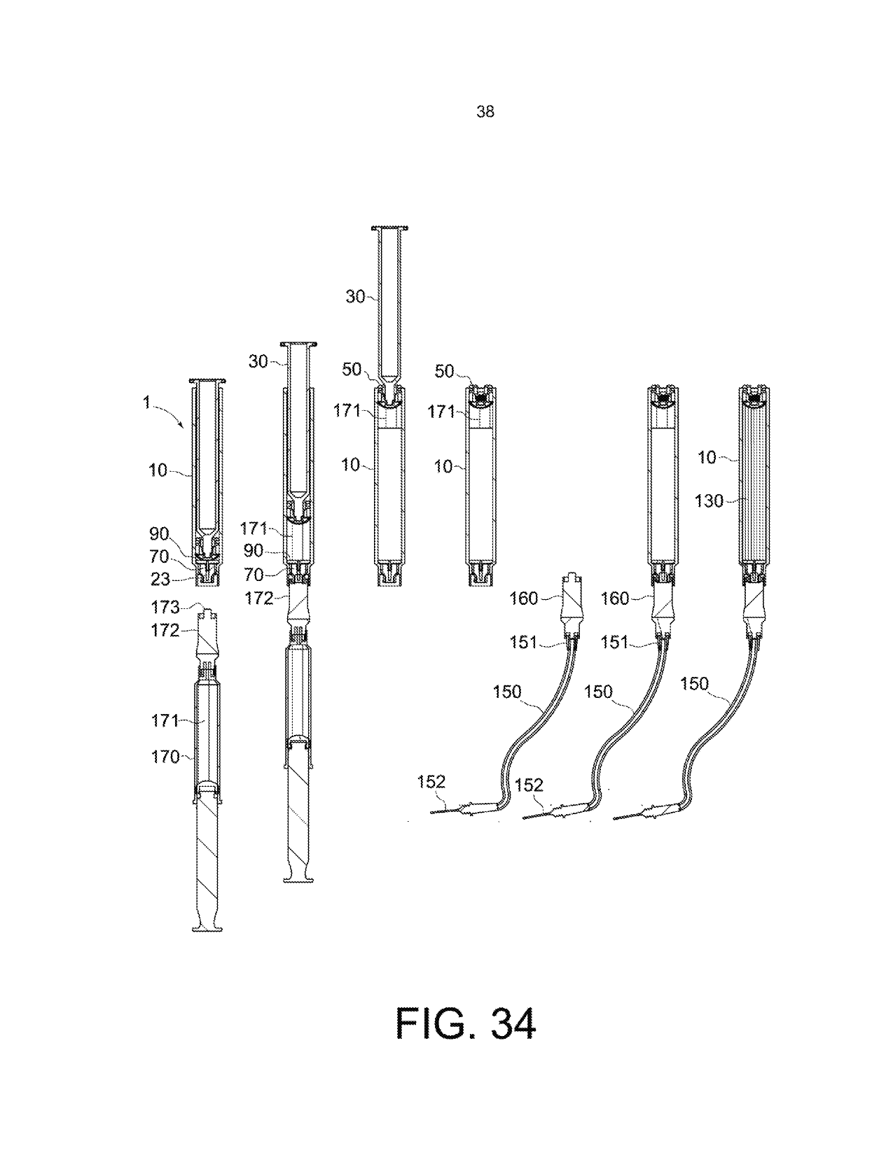

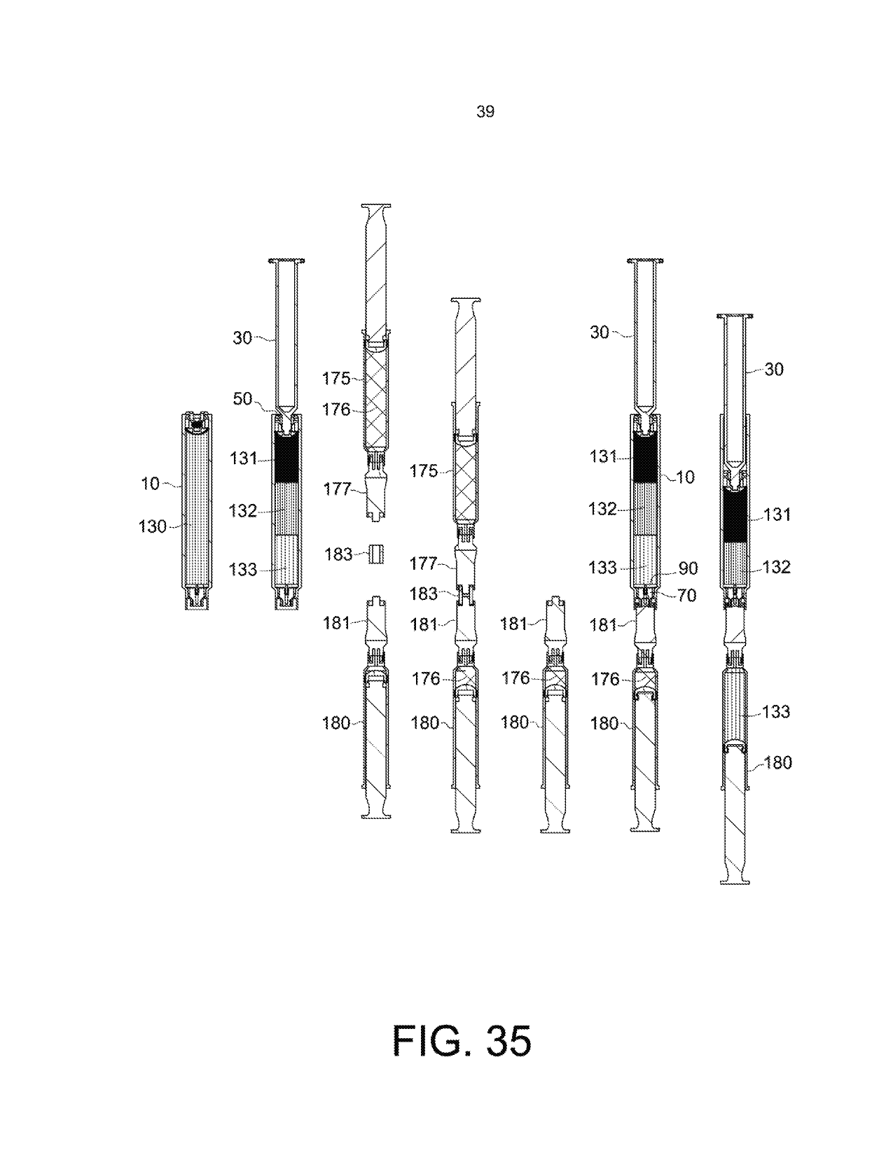

FIGS. 34 and 35 show a sequence of steps in accordance with a further use of the device (1). In a first step of the sequence, the process begins with the compressed tube (10), and an anticoagulant (e.g., sodium citrate) is added to the tube (10). For this purpose, as shown in the first step of FIG. 34, a syringe (170) previously loaded with a certain amount of anticoagulant (171) and having a valve (172) with a male luer-lock connection (173) is used. As shown in the second step, the syringe (170) is connected to the tube (10), thereby opening the valve set (70, 90) and the valve (172), and the required dose of anticoagulant (171) is transferred to the tube (10). The syringe (170) is then removed, and the valve set (70, 90) of the tube (10) automatically shuts closed. Next, as shown in the third step, the handle (30) is pulled to cause a vacuum. When the handle (30) reaches the end of its displacement, the handle (30) becomes clipped to the proximal end (12) of the tube (10). Then, in the fourth step, the handle (30) is removed. Next, blood is hermetically extracted using a butterfly needle (150) as explained with reference to FIG. 32, thereby obtaining a closed, fluid-tight tube (10) containing blood (130) as shown in the first step of FIG. 35. Next, the tube (10) is centrifuged and the handle (30) is reconnected to the piston head (50), as shown in the second step of FIG. 35. In a third step, a first needle (175), preloaded with an activating substance (176) (e.g., calcium chloride) and with a female-male valve (177), is arranged facing a second needle (180), the purpose of which is to receive one or more fractions (131, 132, 133) and which has a female-male valve (181). A female-female connector (183) is disposed between the syringes (175, 180). The valves (177, 181) maintain fluid-tightness inside the respective syringes (175, 180). The transfer takes place in a sterile space and thus the female-female connector (183) is sterile. Next, the first syringe (175), which is preloaded with the activating substance (176), is connected to the fractioning, second syringe (180) and the required dose of activating sequence (176) is transferred to the second syringe (180), as shown in the fourth step. In a fifth step, the first syringe (175), its valve (177) and the female-female connector (183) are disconnected. In a sixth step, the second syringe (180) is connected to the tube (10), and the valve (181) presses and opens the valve set (70, 90) of the tube (10). In a seventh step, fractioning is performed in the second syringe (180) by pressing the handle (30) of the tube (10) until the desired fraction or fractions (131, 132, 133) are delivered to the second syringe (180).

In other words, the method of FIGS. 34 and 35 is the same as the method of FIGS. 32 and 33, with the exception that sodium citrate and calcium chloride are added using preloaded syringes which come with luer connection valves. This can be considered a hermetic circuit, as there is no way its contents can become contaminated; the blood, plasma and surfaces contacted thereby are never exposed to the outside during the manipulation procedure.

In an alternative embodiment, it would be possible to add the additives of the first step of FIG. 34 and the fourth step of FIG. 35 from an ampoule or other container, using a syringe.

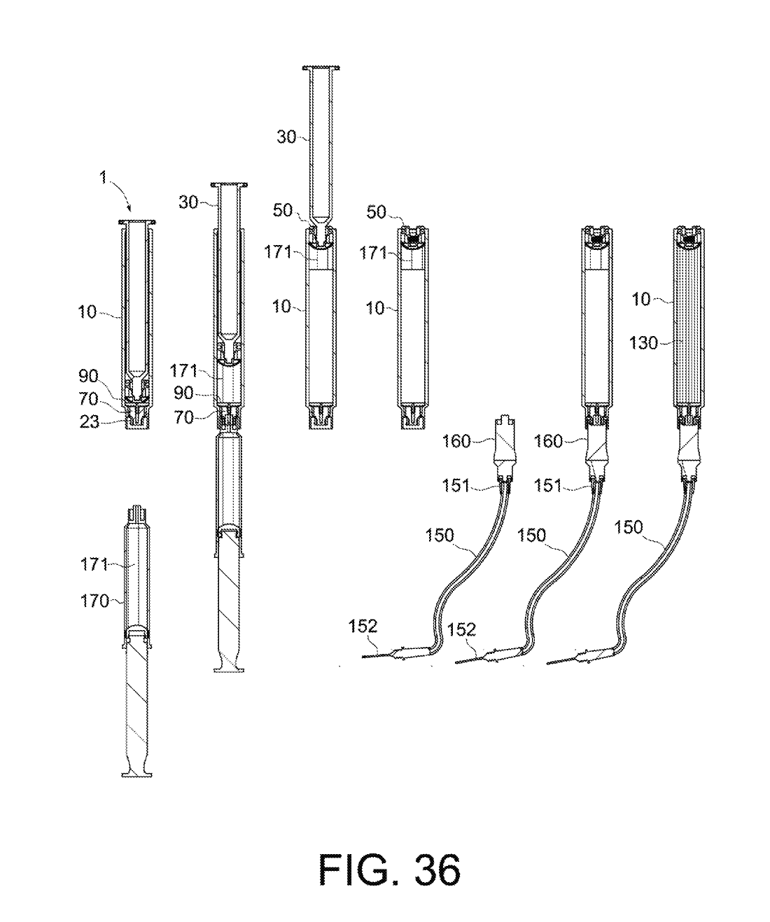

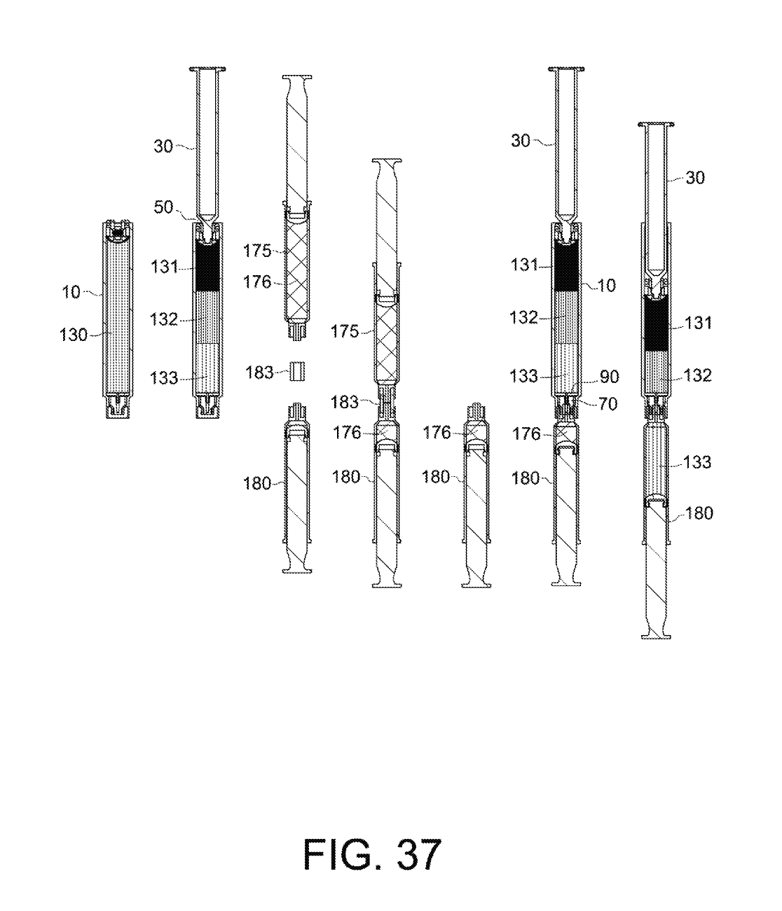

FIGS. 36 and 37 show a sequence of steps in accordance with a further use of the device (1). The sequence is similar to the sequence of FIGS. 34 and 35, but does not use the most compromised valves, i.e. the valve (172) which connects to the syringe (170) containing anticoagulant (171) (FIG. 34), the valve (177) which connects to the first syringe (175) (FIG. 35) and the valve (181) which connects to the fractioning or second syringe (180) (FIG. 35). However, the valve set (70, 90) of the tube (10) remains, keeping the contents of the tube (10) protected.

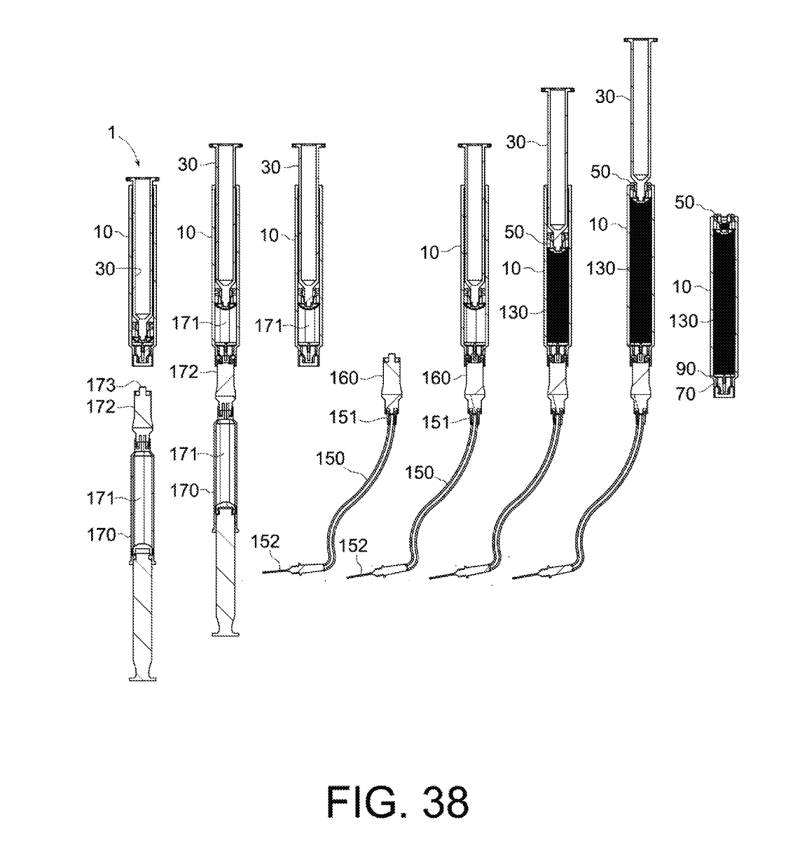

FIG. 38 shows a sequence of steps in accordance with another example of use of the device (1). In this sequence, the tube (10) is operated like a syringe while extracting blood. In a first step, the syringe (170) preloaded with anticoagulant (171) (e.g., sodium citrate) and provided with a valve (172) is obtained, together with the tube (10) in accordance with the invention, arranged in its forwardmost position. In a second step, the syringe (170) with anticoagulant (171) is connected to the tube (10) and the required dose of anticoagulant (171) is transferred to the tube (10). Next, as shown in the third step, the tube (10) is disconnected from the preloaded syringe (170). IN a fourth step, a butterfly needle (150) having a valve (160) with a male outlet is obtained, and the needle (152) of the butterfly needle (150) is inserted into the patient's vein. IN a fifth step, the tube (10) is connected to the valve (160) which is in turn connected to the butterfly needle (150). IN a sixth step, the handle (30) of the tube (10) is slowly pulled so that the blood (130) starts flowing into the tube (10) by suction. The handle (30) continues to be pulled until the piston head (50) reaches the end of its trajectory and becomes clipped to the proximal end of the tube (10), as shown in the seventh step in the figure (note that it would be possible to pull the handle (30) without clipping the piston head (50) and without completely filling the tube (10), which could be of interest in some medical applications). Next, the handle (30) is removed, thereby obtaining a tube (10) filled with blood (130), with the tube (10) fluid-tightly closed by a valve set (70, 90), as shown in the eighth step. The procedure may then continue, for instance, in the same way as other examples described heretofore.

This use of the tube (10), by which blood is suctioned from the vein into the tube (10), allows to better control the manner in which blood is inserted, allowing to prevent hemolysis.

Methods of use of the device (1) are also contemplated which are similar to the aforementioned methods but in which blood is extracted from an IV instead of using a butterfly needle (150).

1. Container device (1) for collecting, storing and processing blood or a blood compound, characterised in that it comprises:
a hollow tube (10), which comprises a tubular body (17) and which delimits an interior space (14) that ends in a proximal opening (15) located in a proximal end (12) of the tube (10) and in a distal opening (16) located in a distal end (13) of the tube (10), wherein the tube (10) further comprises a connector (23) at the distal end (13),
a piston set (30, 50) movably arranged in the interior space (14) of the tube (10) and which comprises a piston head (50) and a handle (30), wherein the piston head (50) is arranged at a distal end of the piston set (30, 50) and contacts the tubular body (17) of the tube (10) delimiting and isolating two regions (14a, 14b) in the interior space (14) of the tube (10), wherein the handle (30) extends from the piston head (50) and protrudes from the tube (10) at the proximal end (12) of the tube (10),
a valve set (70, 90) positioned so as to block the flow of fluid in a distal end of the interior space (14) of the tube (10) and prevent the flow of fluid through the distal opening (16) of the tube (10), wherein the valve set (70, 90) is operable to unblock the flow of fluid through the distal end of the interior space (14) and allow the flow of fluid through the distal opening (16).

2. Device (1), according to claim 1, characterised in that the valve set (70, 90) is operable by a pressure exerted on a distal wall (76) of the valve set (70, 90) towards the proximal end (12) of the tube (10), wherein the pressure produces a deformation of the valve set (70, 90) which causes the formation of a fluid passageway fluid through the valve set (70, 90).

3. Device (1), according to claim 2, characterised in that the wall (76) comprises a through cut (77) that adopts a closed and sealed configuration in the absence of the pressure and an open configuration in the presence of the pressure.

4. Device (1), according to claim 1, characterised in that the valve set (70, 90) is operable by a perforation made by a needle through the valve set (70, 90), wherein the perforation forms a fluid passageway through the valve set (70, 90) and the needle.

5. Device (1), according to claim 1, characterised in that the handle (30) is disconnectable from and reconectable to the piston head (50).

6. Device (1), according to claim 1, characterised in that the handle (30) is connected to the piston head (50) by a threaded union between a threaded connector (39) of the handle (30) and a threaded connector (53) of the piston head (50).

7. Device (1), according to claim 1, characterised in that the piston head (50) is clippable to the proximal end (12) of the tube (10).

8. Device (1), according to claim 7, characterised in that the piston head (50) comprises one or more elastically and radially deformable protrusions (54), which are clippable to the proximal end (12) of the tube (10) to retain the piston head (50) in a position adjacent to the proximal end (12) of the tube (10).

9. Device (1), according to claim 1, characterised in that the handle (30) has a tubular body (34) that is in contact with a lip (25) of the tube (10) on the whole of the perimeter of the tubular body (34) during the displacement of the handle (30) with respect to the tube (10).

10. Device (1), according to claim 9, characterised in that the handle (30) comprises at least two spaces (42) where the lip (25) can be alternatively housed to define discrete positions of the handle (30) with respect to the tube (10), whereby each discrete position corresponds to a different volume from a region (14b) of the device (1) provided with vacuum conditions.

11. Device (1), according to claim 1, characterised in that the connector (23) of the tube (10) is a luer connection.

12. Device (1), according to claim 11, characterised in that the connector (23) of the tube (10) is a male luer connection.

13. Device (1), according to claim 1, characterised in that it further comprises a hood (100) which has an interior space (103) sized to receive the tubular body (17) of the tube (10), and in that it further comprises a neck (105) that extends from a distal end of the hood (100) towards the interior space (103), wherein the neck (105) comprises a threaded connection (106) for the connection of the connector (23) of the tube (10).

14. Device (1), according to claim 1, characterised in that the threaded connection (106) of the hood (100) is a luer connection.

15. Device (1), according to claim 14, characterised in that the threaded connection (106) of the hood (100) is a female luer connection.

16. Device (1), according to claim 1, comprising a hood coupled by friction to the distal end (13) of the tube (10).

17. Device (1), according to claim 1, wherein the tube at the distal end is provided with a skirt (24) which surrounds the connector (23) and protects the connector (23).
